(19) United States
(12) Patent Application Publication
(10) Pub. No.: US 2007/0161830 A1
(43) Pub. Date: Jul. 12, 2007

(54) Process for regenerating a hydrogenation catalyst

(75) Inventor: Michel Strebelle, Brussels (BE)

Correspondence Address: OBLON, SPIVAK, MCCLELLAND, MAIER & NEUSTADT, P.C., 1940 DUKE STREET, ALEXANDRIA, VA, 22314, US

(73) Assignee: SOLVAY (SOCIETE ANONYME), 33 B-1050 BRUXELLES (BE)

(21) Appl. No.: 10/567,263

(22) PCT Filed: Aug. 5, 2004
(86) PCT No.: PCT/EP04/51723

§ 371 Date: Sep. 6, 2006

(51) Int. Cl. C07C 17/156; B01J 38/12

(52) U.S. Cl. 570/245; 502/38

(57) ABSTRACT

Process for regenerating a spent hydrogenation catalyst comprising at least one catalytic metal selected from the group consisting of Ru, Rh, Pd, Os, Ir and Pt on an inert support, the said process essentially consisting of a thermal treatment in the presence of oxygen at a temperature of between 300 and 700° C.

The present invention relates to a process for regenerating a specific hydrogenation catalyst and to an industrial process using such a regenerated catalyst.

Numerous industrial processes employ a catalytic hydrogenation step. Catalysts highly suitable for this purpose are those comprising a metal from group VIII of the Periodic Table, selected from the elements Ru, Rh, Pd, Os, Ir and Pt, on an inert support (silica, alumina, etc.).

An example of such a process is the production of vinyl chloride monomer (VCM) by coupling a direct chlorination and an oxychlorination of ethylene (C₂H₄) to form 1,2-dichloroethane (DCEa), which is subjected to pyrolysis to form VCM on the one hand and HCl on the other. In the course of this pyrolysis a small amount of acetylene (C₂H₂), of the order of approximately 2000 ppm (by volume relative to the volume of HCl), is co-produced, but cannot easily be separated from the HCl, owing to their very similar volatilities. The pyrolysis HCl is then recycled to the oxychlorination, in the course of which the C₂H₂ reacts to give various worthless by-products, which are detrimental to the profitability of the process. One known method, an elegant one, for removing this C₂H₂ consists in converting it into ethylene (C₂H₄) by hydrogenation, using an appropriate catalyst. One such catalyst is described in patent application DE 24 38 153, which illustrates in particular a catalyst based on Pd supported on non-porous silica. In service, however, this catalyst undergoes gradual deactivation and, although the abovementioned application records the possibility in theory of regenerating it, in practice such regeneration has proved to be fruitless, owing in particular to the contamination of this catalyst with heavy metals (H. Müller et al., Chem.-Ing.-Tech. 59 (1987) No. 8, pp. 645-7).

The applicant, however, has surprisingly found that if such a contaminated catalyst is treated in the presence of oxygen, at a temperature sufficient to remove the contaminations but not too high, so as not to impair the catalyst, the said catalyst can nevertheless be regenerated satisfactorily.

The present invention accordingly provides a process for regenerating a hydrogenation catalyst comprising at least one catalytic metal selected from the group consisting of Ru, Rh, Pd, Os, Ir and Pt on an inert support, the said regeneration process consisting essentially of a thermal treatment in the presence of oxygen at a temperature between 300 and 700° C.

Of the aforementioned catalytic metals preference is given to Pt and Pd. Pd is particularly preferred on account of its high hydrogen adsorption capacity. The concentration of the catalytic metal in the catalyst is generally greater than or equal to 0.01% by weight (relative to the total weight of the catalyst), preferably greater than or equal to 0.05%, or even greater than or equal to 0.1%. This concentration is, however, generally less than or equal to 10%, or even less than or equal to 5%, or even less than or equal to 1%.

The inert support of the catalyst which is regenerable by the process according to the present invention is preferably selected from porous and non-porous silica, alumina and silica-alumina. Supports based primarily on silica (in other words composed of more than 50%, preferably of more than 95%, of SiO₂) give good results. The support is preferably non-porous or of low porosity, in other words having a specific surface area (measured in accordance with the BET method with nitrogen) of less than 5 m²/g, and preferably less than 3 m²/g, or even less than 1 m²/g. The average pore volume of this support is advantageously less than 0.01 ml/g. Its particle size is advantageously between 1 and 20 mm, or even between 2 and 10 mm, and preferably between 3 and 7 mm. On this support the catalytic metal is generally present in a layer of less than or equal to a micron. It is generally in the form of crystallites having a size of between 0.1 and 0.5 μm. In particular the non-porous silica as described in the aforementioned references (DE 24 38 153 and the article by Müller) gives good results.

By the fact that the process according to the invention “essentially consists of a thermal treatment” is meant that the major part of the regeneration of the catalyst (in other words at least 50% of the gain in selectivity and/or in degree of conversion) is realized by the thermal treatment. Preferably at least 75% of the regeneration is the outcome of the thermal treatment, or even at least 90%, and with particular preference the entirety of the regeneration is the outcome thereof, implying that according to this version of the invention the process takes place in the absence of any regenerative treatment (with steam or H₂, for example) preceding or following the said thermal treatment, and therefore that the catalyst obtained from the thermal treatment is reused as it is in a hydrogenation reaction.

However, care is generally taken to remove the reactants still present on the surface of the catalyst (by flushing with nitrogen, for example) before the thermal regeneration according to the invention.

Similarly, by “reuse of the catalyst as it is” is meant a use identical to that of a fresh catalyst. Such use may include, for example, prior activation by flushing with H₂.

The thermal treatment in question consists in a residence at a high temperature (of between 300 and 700° C.) in the presence of oxygen. The temperature during the thermal treatment is preferably greater than or equal to 400° C., or even greater than or equal to 500° C., in order to increase the efficiency of the regeneration. It is, however, preferably less than or equal to 600° C., or even less than or equal to 550° C., so as not to impair the catalyst (since it is known that, at too high a temperature, supported catalysts may undergo “fritting”, or agglomeration of the catalytic metal, resulting in a loss of activity by reduction of the active surface). The thermal treatment may take place in the presence of pure oxygen. Preferably, however, the oxygen is diluted, with an inert gas for example. Accordingly air gives good results.

The treatment in question therefore in fact typically involves what is generally referred to as an oxidizing atmosphere, which may be either static or moving (which is to say that a gaseous stream containing oxygen is passed over the catalyst to be regenerated). A moving oxidizing atmosphere gives good results. A simple residence in a stove or electric oven, preferably with a fan, may serve for thermal treatment according to the present invention. Another way which gives good results consists in passing the oxidizing atmosphere through the bed of catalyst, in situ for example, in the hydrogenation reactor.

Better results are generally obtained when the catalyst is dispersed during the treatment: that is, when it presents a maximum surface area to the oxidizing atmosphere. Hence the catalyst will advantageously be spread in a layer, ranging from a monolayer of catalyst (whose thickness depends on the particle size of the catalyst) to a layer of approximately 20 cm, although, preferably, the thickness of this layer does not exceed 10 cm, or even 5 cm.

The duration of the said treatment is readily determined by the skilled person and will be adapted to the desired degree of regeneration. It is generally greater than or equal to 1 h, or even to 5 h. This duration is, however, generally less than or equal to 48 h, or even to 24 h. The same applies to the ventilation flow rate, which is preferably greater than or equal to 0.01 l/min.kg cata (or litre per minute per kg of catalyst), or even greater than or equal to 0.1 l/min.kg cata, but is generally less than or equal to 100 l/min.kg cata, or even less than or equal to 10 l/min.kg cata.

The catalyst that it is intended should be regenerated by the process according to the invention is a “spent” catalyst, (i.e. a catalyst which has served in a hydrogenation reaction) subsequent to which its catalytic activity (in terms of selectivity and/or degree of conversion) has dropped. Such a drop in catalytic activity is generally ascribed to the deposition of carbonaceous substances and/or to contamination with chlorine compounds and/or traces of at least one heavy metal. The term “heavy metal” is intended to denote one of the following metals: Al, As, Cd, Cr, Ni, Cu, Sn, Fe, Mn, Hg, Pb, Zn and Ti (although the latter is not generally considered to be a heavy metal, it nevertheless constitutes a disruptive contamination for hydrogenation catalysts and, as such, is considered to be a heavy metal in the context of the present invention). The traces of heavy metals are particularly disruptive and, among them, Fe and Ti in particular, since they are commonly present in industrial fluids, owing to the nature of the equipment used to convey/treat them. Similarly, traces of Hg, which may be encountered in certain sources of H₂, are also disruptive. By “traces” are meant amounts of the order of ppm, or even tens of ppm. It is not uncommon for the starting catalyst already to include traces of certain heavy metals (Fe in particular, but generally less than 50 ppm), but in the course of use an increase in the amount thereof (for example to an amount greater than or equal to 50 ppm in the case of Fe) generally contributes to a drop in the catalytic activity.

The hydrogenation reaction in which the catalyst has been used is preferably an acetylene hydrogenation reaction. It applies preferably to traces of acetylene (C₂H₂) which are present in a fluid and, preferably, in a gas mixture consisting essentially of HCl and obtained from the pyrolysis of DCEa, as described above. Such a mixture generally contains between 1500 and 2500 ppm of acetylene. It often also contains of the order of tens to hundreds of ppm of chlorinated organic products such as VCM and methyl or ethyl chloride, and/or non-chlorinated organic products such as ethylene (C₂H₄), methane and butadiene. These contaminants result from imperfect separation during operations to separate pyrolysis products from HCl, the said separation generally being carried out by distillation. For this type of reaction, as described above, catalysts based on Pd on a non-porous silica support give good results and are readily regenerable by the process according to the invention.

The catalyst regenerated by the process according to the invention may be used in any hydrogenation reaction for which it has a catalytic activity. Preferably it is reused in a process similar to that in which it was used beforehand. Thus the present invention likewise provides a process for synthesizing VCM by coupling of a direct chlorination and an oxychlorination of ethylene to form DCEa, which is primarily converted into VCM and into HCl by pyrolysis, the said HCl containing traces of acetylene and being recycled to the oxychlorination following hydrogenation of these traces of acetylene in the presence of a catalyst regenerated by a process as described above.

The present invention is illustrated non-limitatively by the following example:

Catalyst E39H (beads of silica 3 to 5 mm in diameter with 0.15% of Pd supported at the surface and with a specific surface area of less than 1 m²/g), sold by Degussa and as described in the aforementioned article by Müller, was used for four and a half years (54 months) in contact with HCl containing approximately 2000 ppm of C₂H₂ under 10 bars and at a temperature of between 120 and 180° C. The residence time (ratio between the number of m³ (s.t.p.) of HCl/h and the volume of the catalyst bed in m³) was 1680 h⁻¹. The quantity of H₂ employed was 3.8 mol per mole of C₂H₂.

The thus-spent catalyst was analyzed and compared with the virgin catalyst. The results of these analyses are given in the table below:

| Element analyzed | | Spent |
|---|---|---|
| (content) | Fresh catalyst | catalyst |
| Pd (weight %) | 0.15 | 0.14 |
| Cl (weight %) | 0.015 | 1.090 |
| Fe (ppm) | <50 | 84 |
| Traces | — | Co, Zn, Cu, Ti, Pb, Zr |

A batch of 150 kg of this spent catalyst was spread over 18 plates each with a surface area of 0.3 m². The temperature of the oven was taken to 500° C. and held for 18 h. The ventilation of the oven is controlled by an air input of 100 l/min.

This batch was subsequently reused under conditions similar to those described above, at a temperature of 173° C., and its catalytic activity was compared with that of the spent catalyst at end of life (used at 180° C.) in the following table:

| | Spent | Regenerated |
|---|---|---|
| | catalyst | catalyst |
| Degree of conversion of C₂H₂ | 82 | 94.6 |
| (%) | | |
| Yield (molar % C₂H₄/C₂H₂) | 48.7 | 62.9 |

It is found that the catalytic activity was highly regenerated (improved conversion and improved yield despite the lower operating temperature).

1-9. (canceled)

10. A process for regenerating a spent hydrogenation catalyst comprising at least one catalytic metal selected from the group consisting of Ru, Rh, Pd, Os, Ir and Pt on an inert support, wherein the spent catalyst has been used in a reaction of hydrogenation of traces of acetylene which are present in a gas mixture consisting essentially of HCl and obtained from the pyrolysis of 1,2-dichloroethane (DECa) and in that the said process consists essentially of a thermal treatment in the presence of oxygen at a temperature of between 300 and 700° C.

11. The process according to claim 10, wherein the catalytic metal is Pd.

12. The process according to claim 10, wherein the inert support is based primarily on silica.

13. The process according to claim 10, wherein the inert support has a BET surface area of less than 5 m²/g.

14. The process according to claim 10, wherein the temperature during the thermal treatment is between 400 and 600° C.

15. The process according to claim 10, wherein the thermal treatment takes place in the presence of air.

16. The process according to claim 10, wherein the thermal treatment consists in a residence in a stove or a ventilated electric oven.

17. The process according to claim 10, wherein the catalyst is contaminated with traces of heavy metals.

18. A process for synthesizing vinyl chloride monomer (VCM) by coupling a direct chlorination and an oxychlorination of ethylene to form DCEa, which is converted primarily into VCM and into HCl by pyrolysis, the said HCl containing traces of acetylene and being recycled to the oxychlorination following hydrogenation of these traces of acetylene in the presence of a catalyst regenerated by the process according to claim 10.
